(19) United States
(12) Patent Application Publication
(10) Pub. No.: US 2005/0261649 A1
(43) Pub. Date: Nov. 24, 2005

(54) Absorbent article with layered acquisition/distribution system

(76) Inventor: Richmond R. Cohen, Williamsport, PA (US)

Correspondence Address: AMSTER, ROTHSTEIN & EBENSTEIN LLP, 90 PARK AVENUE, NEW YORK, NY, 10016, US

(21) Appl. No.: 10/849,460

(22) Filed: May 19, 2004

(51) Int. Cl. A61F 13/15

(52) U.S. Cl. 604/383

(57) ABSTRACT

An absorbent article has a topsheet, an absorbent core, and a layered acquisition/distribution transfer system disposed intermediate the topsheet and the absorbent core. The system has at least a pair of apertured films including a first film facing the topsheet and a second film facing the absorbent core. Each film is three-dimensional, formed of a wettable and substantially non-absorbent thermoplastic polymer, and defines pores which taper inwardly in a first direction from the topsheet to the absorbent core. The first film has a larger average pore size than the second film.

### BACKGROUND OF THE INVENTION

The present invention relates to absorbent articles generally, and more particularly to such articles having an acquisition/distribution system (ADS) disposed intermediate a topsheet and an absorbent core.

Disposable absorbent articles such as baby diapers, adult diapers, and feminine hygiene products today typically have multiple layers of absorbent materials or composites. The articles always have a topsheet and an absorbent core. The absorbent core is generally a composite of fluff pulp and superabsorbent polymer (SAP) that stores most of the liquid entering the article. Most diapers also contain an acquisition/distribution layer (ADL) interposed between the topsheet and the absorbent core. The functions of the ADL include improvement of the rate of liquid uptake into the diaper (i.e., increase the liquid acquisition speed), improvement in the retention of liquid in the diaper (i.e., lower the rewet or wetback characteristics), and improvement in spreading the liquid throughout the diaper to utilize its capacity more effectively (i.e., the distribution factor which affects both the acquisition rate and rewet characteristics).

As discreetness is an important issue for many wearers of absorbent products, diapers that are termed “thin” are becoming more prevalent. Generally, these diapers are rendered thin by replacing a significant percentage of the fluff pulp in the absorbent core with SAP and then compressing the absorbent core. Although such techniques are effective in providing a thinner diaper, the absorbent properties of the diaper may be compromised. With the combination of compression and increased SAP content, thin diapers tend to show slow speeds of liquid acquisition and reduced wicking and spreading of liquid. As a result, such structures are more prone to leakage. Such is the case regardless of the properties of the SAP. Hence, the enhancement in discreetness, comfort and fit developed by making a thin structure may be offset by poor absorbency—i.e., slow acquisition and high rewet.

The increased tendency of thin diapers to leak places greater importance on the ADL to perform better. One means of gaining greater performance is through increasing the basis weight of the ADL. With an incrementally heavier ADL, the resulting performance of the diaper will be improved incrementally. Other types of absorbent structures designed to improve the rate of absorbency, while inhibiting wetback or rewet, involve multiple layers of fibrous webs having strategically varied average pore sizes (U.S. Pat. No. 5,728,083, U.S. Pat. No. 5,569,226, and U.S. Pat. No. 5,505,719 to Cohen, et al.).

Combinations of three-dimensional apertured film with fibrous webs have also been tried to improve absorbent properties (U.S. Pat. No. 6,455,753, and U.S. Pat. No. 5,603,707). Such absorbent structures are claimed to be efficacious in the absorbent article. However, to produce an absorbent article containing these layers of different materials with different elongational properties may be cumbersome to manufacture inline.

Thus, the need remains for an ADL that can perform better in conjunction with a thin absorbent core. Such an ADL structure would exhibit a special synergy with the thin absorbent core, meaning that it would simultaneously improve the ability of the core to absorb faster, to retain liquid better and to enhance the spreading and wicking of liquid. Such an ADL structure must also be convenient and practical to manufacture in an absorbent article.

The material interposed between the topsheet and the absorbent core ideally acts as an acquisition/distribution layer which receives the liquid from the topsheet and distributes it laterally before it enters the absorbent core. This distribution of liquid prevents over-saturation of a local area of the absorbent core by increasing the surface area of the core receiving the liquid. Being well-distributed, the liquid from the ADL is better absorbed by the absorbent core because it avoids the formation of liquid pools in an over-saturated local area of the absorbent core. Thus the acquisition/distribution layer not only improves strike-through (the time required to absorb the liquid insult), but also improves rewet characteristics (that is, the amount of liquid which leaks back from the absorbent core through the acquisition/distribution layer).

The importance of the acquisition/distribution layer becomes more evident with subsequent liquid insults directed to the same local area of the core as the local area tends to already be filled with liquid from the previous liquid insult. The difficulty in wicking or distribution of the initial liquid insult leaves the local area of the core already wet and thus less capable of handling subsequent liquid insults.

It is recognized that nonwovens provide a high level of distribution when used as an acquisition/distribution layer, so as to provide for good horizontal spreading or distribution of the liquid insult. However, the performance of nonwovens is known to be limited within certain boundaries. The kind of properties needed for nonwovens to provide faster acquisition speeds often limit the layer's ability to inhibit rewet, promote wicking and enable efficient processing.

Accordingly, it is an object of the present invention to provide an absorbent article having an improved acquisition/distribution system which, in a preferred embodiment, provides improved acquisition times (strike-through times) and improved rewet characteristics.

Another object is to provide such an article which, in a preferred embodiment, is characterized by reduced run-off (that is, reduced leakage) and improved surface dryness characteristics.

A further object is to provide such an article wherein, in a preferred embodiment, the acquisition/distribution structure does not introduce processing problems during inline production of the article.

It is also an object of the present invention to provide such an article which, in a preferred embodiment, is simple, inexpensive and easy to manufacture, use and maintain.

### SUMMARY OF THE INVENTION

It has now been found that the above and related objects of the present invention are obtained in an absorbent article comprising a topsheet, an absorbent core, and a layered acquisition/distribution transfer system disposed intermediate the topsheet and the absorbent core. The acquisition/distribution system (ADS) comprises at least a pair of apertured films including a first film facing the topsheet and a second film facing the absorbent core. Each film is three dimensional and defines pores which taper inwardly in a first direction from the topsheet to the absorbent core, with the first film having a larger average pore size than the second film. The pores terminate in substantially thick projections in the first direction beyond a main plane of the film.

Preferably, the thickness of the projections is on average at least 40, optimally at least 50-100, times that of the main plane of the film

Preferably the films have a combined loft or thickness of at least 30 mils (0.76 mm), optimally at least 50 mils, and are formed of substantially the same polymer, e.g., polyethylene. The first and second films are contiguous and preferably laminated together. The pores of each film are generally conical. Each film is formed of a wettable and substantially non-absorbent thermoplastic polymer.

In a preferred embodiment, the first film has an average pore size of 0.3-10.0 mm in diameter (preferably 0.5-5.0 mm and optimally 1.0-2.0 mm), and the second film has an average pore size of 0.1-2.0 mm in diameter (preferably 0.3-1.5 mm and optimally 0.5-1.0 mm). The first film has a basis weight at least as high as the second film, the first film having a basis weight of 25-100 gsm (preferably 30-65 gsm and optimally 35-50 gsm), and the second film having a basis weight of 10-35 gsm (preferably 15-30 gsm and optimally 20-30 gsm).

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the accompanying drawing wherein:

FIG. 1 is a top plan view of an absorbent article according to the present invention in a stretched-out orientation;

FIG. 2 is an exploded sectional view thereof taken along the line 2-2 of FIG. 1, with each layer being schematically indicated and a circled portion of each film of the ADS being shown to an enlarged scale; and

FIG. 3 is a fragmentary sectional view, to an enlarged scale, of the novel acquisition/distribution system of the present invention.

### DETAILED DESCRIPTION OF THE PREFERRED EMBODIMENTS

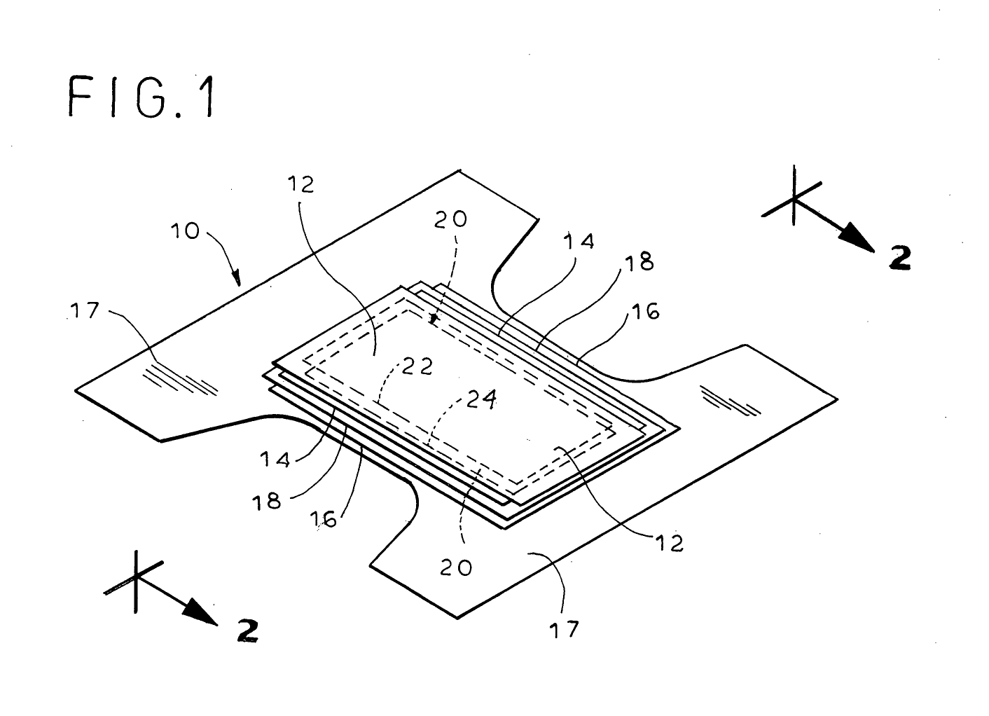

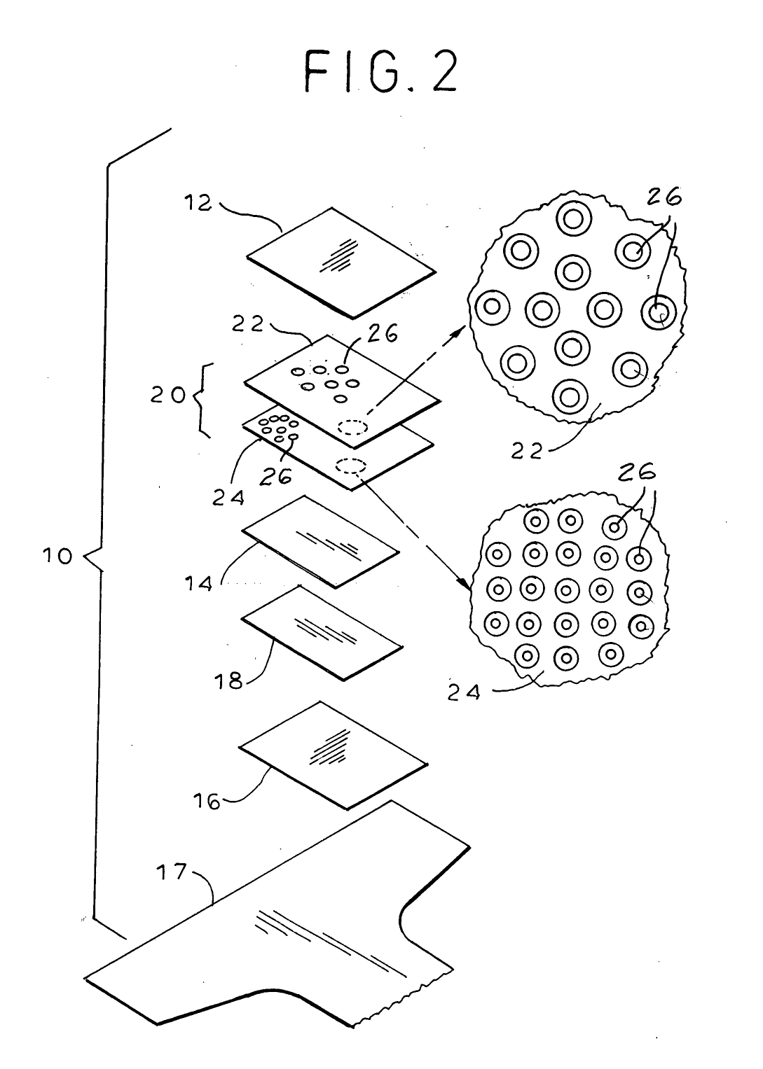

Referring now to the drawing, and in particular to FIGS. 1 and 2 thereof, therein illustrated is an absorbent article according to the present invention, generally designated by the reference numeral 10. Basically, the absorbent article 10 includes a skin-facing liquid-pervious topsheet 12 (typically a nonwoven), an absorbent core 14 (which typically includes fluff pulp and SAP) for absorbing a liquid insult, and a garment-facing liquid-impervious backsheet 16. The article 10 may include a chassis 17 and may contain a tissue 18 disposed intermediate the core and the backsheet to assist in maintaining the ingredients of the core 14 together, crotch elastics (not shown) and the like.

According to the present invention, a layered acquisition/distribution transfer system, generally designated 20, is disposed intermediate the topsheet 12 and the absorbent core 14. More particularly, the layered acquisition/distribution system (ADS) 20 includes at least a pair of flexible apertured films 22, 24, including a first film 22 facing the topsheet 12 and a second film 24 facing the absorbent core 14. Each film 22, 24 is three dimensional (as opposed to flat) and preferably formed of a wettable (i.e., hydrophilic) but substantially non-absorbent thermoplastic polymer such as the polyolefins (e.g., polyethylene, polypropylene and the like) that are rendered wettable (hydrophilic) through treatment, for instance, through topical addition or internal addition of a surfactant. Preferably films 22, 24 are formed of substantially the same polymer, thereby to provide substantially the same elongational properties and thus facilitate inline processing. The films 22, 24 of the ADS have a combined loft or thickness of at least 30 mil (0.76 mm) and preferably at least 50 mils (1.3 mm). Thickness is measured by a Digital Micrometer, Model 49-72, available from Testing Machines, Inc. (with a 2 inch diameter anvil for applying a load of 95 g/in² to the sample). Preferably the acquisition/distribution system 20 has a density not exceeding 0.07 g/cc so as to provide sufficient loft or thickness to the system.

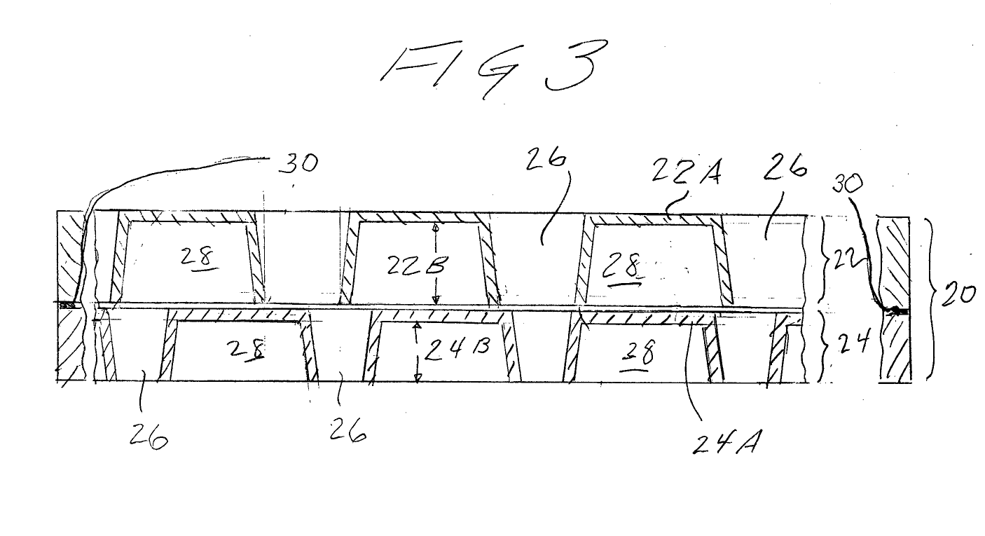

Referring now to FIG. 3 as well, the films 22, 24 are characterized by apertures or pores 26 therethrough sufficiently large to enable rapid liquid acquisition. The apertures or pores 26 may be created during film formation or thereafter. Each film 22, 24 defines pores 26 which taper inwardly in a first direction from the topsheet 12 to the absorbent core 14. Preferably the pores are conically shaped, with the hollow cones projecting (that is, tapering inwardly) in the first direction (i.e., towards the core 14) to inhibit the reverse flow of liquid (i.e., rewet or liquid flow towards the topsheet 12). Preferably, the tapered cones are truncated and have the maximum and minimum openings of the cones decreasing in size as the liquid travels therethrough in the first direction from the first film 22 (adjacent the topsheet 12) toward the second film 24 (adjacent the core 14). Alternatively, the pores 26 may be polygonal or irregularly shaped, although truncated cones are preferred.

The first film 22 has a larger average pore size than the second film 24. Additional layers of apertured films (not shown) may be disposed intermediate the second film 24 and the core 14 with these additional layers optionally continuing the gradient in pore size established by the first two layers of apertured films 22, 24. While logically it would appear that any additional layers of apertured films should continue the gradient in pore size established by the first two apertured layers of film 22, 24, in view of the surprising experimental results described in the three aforementioned patents to Cohen, et al., in the context of fibrous layers, the continuation of the gradient in pore size through the additional layers may not be necessary (or even desirable) to achieve the purposes of the present invention.

As earlier indicated, the average pore size of the first film 22 is larger than the average pore size of the second film 24. Thus, in a preferred embodiment, the first film has an average pore size of 0.3-10.0 mm in diameter (preferably 0.5-5.0 mm and optimally 1.0-2.0 m), while the second film 24 has an average pore size of 0.1-2.0 mm in diameter (preferably 0.3-1.5 mm and optimally 0.5-1.0 mm).

In a preferred embodiment, the first film 22 has a basis weight at least as high as the second film 24. Thus, the first film has a basis weight of 25-100 gsm (preferably 30-65 gsm and optimally 35-50 gsm), while the second film has a basis weight of 10-35 gsm (preferably 15-30 gsm and optimally 20-30 gsm). Where there are additional films between the second film 24 and the core 14, preferably the basis weight of the first film 22 is at least as high as that of the second film 24 and any additional films.

The pores 26 of each film 22, 24 may be formed by conventional means well-known in the art. One preferred technique involves the use of heat and suction. Thus each generally planar film is heated to its softening point (below the melting point), and then suction is applied to the bottom side of the film to form the pores 26. The suction draws the material of the film downwardly, typically through an apertured carrier or screen supporting the film, so that pores of the desired configuration are formed within the film. In this preferred technique of forming the pores 26, at least a portion of the material drawn out of the main plane of the film by the suction remains a part of the film and projects downwardly below the main plane of the film as hollow projections 28. The projections 28 are on average at least 40, preferably at least 50-100, times greater in thickness than the main plane of the respective films 22, 24 and thus preferably provide about 95% of the total loft of the films, the remaining 5% being provided by the main plane of the films. The film thickness measurements provided hereinbelow and in the Example include the projections 28.

Upon subsequent assembly of the ADS, the bottoms of the downward projections 28 of first film 22 contact and locally space the main plane 22a of the first film 22 above the main plane 24a of the second film 24 by a thickness 22b. The presence of the projections 28 desirably increases the overall loft or thickness of the ADS and, in particular, creates laterally extending channels intermediate the bottom of the main plane of the first film 22 and the top of the main plane of the second film 24 so that liquid can easily pass laterally between the films 22, 24. Thus, liquid which passes through a pore 26 of the first film 22 and emerges therefrom to find no pore 26 of the second film 24 directly therebelow, is able to travel laterally through such channels until it finds an adjacent pore 26 of the second film 24 into which it can enter, thereby avoiding a local bulking of the liquid between the films 22, 24.

The projections 28 extending downwardly from the main plane 24a of the second film 24 play a similar role in spacing the main plane 24a of the second film 24 above the top of the absorbent core 14 by a thickness 24b and thereby creating laterally extending channels, in this case between the bottom of the main plane 24 a of the second film 24 and the top of the absorbent core 14. Thus, liquid passing downwardly through pores 26 of the second film 24 and not being immediately absorbed by an aligned portion of the core 14 therebelow will laterally traverse such channels until they reach a portion of the absorbent core 14 which is capable of absorbing the liquid, this time to avoid a local bulking of the liquid intermediate the film 24 and the absorbent core 14.

Preferably, the first film 22 has a main plane thickness of 1-3 mils and a projection thickness 22b of at least 40 mils, optimally at least 50-100 mils, while the second film 24 has a main plane thickness of 0.5-1.5 mils and a projection thickness 24b of at least 25 mils, optimally 10-40 mils. Thus the laterally extending channels between the first and second films 22, 24 are preferably thicker than those between the second film 24 and the absorbent core 14. It will be appreciated that the projections 28 are formed by relatively thin walls, which preferably, but not necessarily, continue the taper of pores 26. The thin walls form only loose contacts with the surface below (whether it be the main plane 24a or the top of the absorbent core 14) so that the liquid can pass through the loose contacts and enter into the laterally extending channels.

The first and second films 22, 24 are thus generally contiguous and optionally peripherally laminated or otherwise secured together by conventional means such as adhesive bonding 30, heat bonding, ultrasonic bonding or other means well known in the art. Alternatively, however, the films 22, 24 are simply juxtaposed without actually being secured one to the other, the overall structure of the absorbent article maintaining them in appropriate juxtaposition. Where the layered system 20 is to be laminated, the lamination process may occur inline or offline, with the appropriate adhesive 30 injected between the peripheries of the two films 22, 24. Either a roll of the laminated goods or individual rolls of the two films (with or without adhesive 30 injected between the longitudinal edges thereof) may be layered between the topsheet 12 and the absorbent core 14 of the absorbent article 10 during manufacture thereof, as described hereinabove. Because the films 22, 24 have generally similar elongational and tensile properties, inline formation and registration of the multilayer ADS structure is less arduous than if the materials had different extensional behaviors.

Where the films 22, 24 are naturally hydrophobic, the films must be treated with one or more wetting agents to render them suitable for use in the layered acquisition/distribution system of the present invention. The wetting agents may be topically applied to the films or present in the form of an internal additive. It is important that the wetting agents impart a durable hydrophilicity to the films 22, 24 so that the films are able to withstand repeated “liquid insults” while still maintaining their hydrophilicity. In other words, it is essential that not all of the wetting agent wash off the films during the first insult.

The unique structure of the ADS of the present invention, when used in the body of an absorbent article, yields a better combination of acquisition speed and rewet values than do the individual film layers comprising the ADS, alternate combinations of these individual film layers, or conventional nonwoven ADL materials. The benefits of the present invention are particularly evident with a thin absorbent core because such thin absorbent cores typically contain high percentages of SAP and therefore tend to suffer from poor acquisition speeds. Hence, the ADS has a synergistic effect with a thin core, yielding a discreet absorbent article particularly resistant to leakage.

The efficacy of the present invention was measured using the following test procedures.

Test Methods

The test procedure used to evaluate the performance of the invention measures the acquisition time and rewet of an absorbent structure for multiple insults. The procedure is similar to others that are widely used in the field.

The absorbent structure is laid flat on a surface (leg gathers are trimmed, if applicable, to accomplish this). A dosing ring (60 mm I.D., 70 mm O.D., and 40 mm height) is placed on the targeted area of the absorbent structure.

Then, 100 ml of synthetic urine (0.9% NaCl solution) is measured in a graduated cylinder and poured into a 125-ml separatory funnel. The funnel discharges liquid at a rate of 9 ml/s when its stopcock valve is opened fully. Positioning the bottom tip of the funnel 40 mm from the surface of the absorbent structure in the center of the dosing ring, the stopcock is opened, and liquid is dispensed onto the absorbent structure. Simultaneously, a timer is activated. The timer is stopped when the 100-ml dose completely passes through the topsheet. This time is recorded as the first acquisition time, in seconds.

The dosing ring is now removed and another timer is activated to measure 15 minutes. After 15 minutes, a stack of pre-weighed filter paper (AFI Grade 950, 9-cm diameter) weighing about 10 g is placed in the center of the wetted target area. A cylindrical weight applying 1 psi of pressure is placed on top of the filter paper, with the weight having a diameter also of 9 cm. After waiting 1 minute, the weight is removed, and the filter paper is weighed. The difference in weight is recorded as the first rewet amount, in grams (g).

Two additional 100-ml doses of synthetic urine are applied using almost the identical procedure as outlined above to produce a total of three “insults” per absorbent structure. For the second and third insults, 15 g of filter paper is used (rather than 10 g). The total number of replications is either 5 or 10 per absorbent structure, with the average values of the acquisition times and rewet amounts computed.

### EXAMPLE

Absorbent structures were prepared comprising in sequence:
a 13.5 gsm liquid-permeable nonwoven topsheet of polypropylene spunbond nonwoven (0.150 mm thick) available under the trade name SB 1350021 from First Quality Nonwovens,
an ADL or ADS,
a 300 gsm thin absorbent core of cellulose fluff and SAP (about 50:50 ratio), laminated with tissue on the back, available under the trade name NOVATHIN from Rayonier, Inc., and
a liquid-impermeable film backsheet of polyethylene (1.1 mm thick) available under the trade name DH-203 from Clopay Plastic Products.

The absorbent core and topsheet are cut to 21″ long and 5.75″ wide. The ADL or ADS is cut to 21″ long and 3.25″ wide.

The materials for the ADL or ADS include:
NW: polyester nonwoven of 30 or 50 gsm available under the trade names 9325642 and 9342736, respectively, from BBA Nonwovens,
AD: a 50 mil thick apertured polyethylene film of 36 gsm with conical pores, available under the trade name AQUIDRY from Tredegar Film Products, and
DW: an 18 mil thick apertured polyethylene film of 24 gsm (with smaller conical pores than AD) available under the trade name #25475 from Tredegar Film Products.

The accompanying Table contains the performance data.
In the Table, acquisition/distribution materials are designated by individual codes or, when combined into an ADS, the layer listed first (that is, prior to the “+”) is the layer that is facing the topsheet.
From the Table, it is evident that the absorbent structures that showed the lowest acquisition times and the lowest rewets are the “AD+DW” ADS of the present invention (data is in the second-to-last row of the Table) and the “AD+30 gsm NW” combined structure ADS involving the apertured film AD and the 30 gsm nonwoven NW (data is in the fifth row of the Table). Between the two absorbent structures showing the lowest acquisition times and the lowest rewets, the ADS of the present invention is preferable to the combined AD+NW structure due to the relative ease of inline processing of the former.

Especially in terms of multiple insults (i.e., the second and third insults) and especially in terms of the rewet characteristics, the ADS of the present invention was superior to both the reversed “DW+AD” ADS and the “AD+AD” ADS (where both films were of the same average pore size).

The poorest performances are shown when the nonwovens alone are used as the ADL.

The present invention has utility in a wide range of absorbent articles including, by way of example, adult briefs, protective underwear, baby diapers, pull-ups and the like. The present invention is also useful with sanitary elasticized male guards of the type described in U.S. patent application Publication No. 2003/0149412 (published Aug. 7, 2003) and loopless absorbent articles of the type described in U.S. patent application Publication No. 2003/0220626, published Nov. 27, 2003.

To summarize, the present invention provides an absorbent article having an improved acquisition/distribution system which provides improved acquisition times (strike-through times) and improved rewet characteristics, the article being characterized by reduced run-off (that is, reduced leakage) and improved surface dryness characteristics. The acquisition/distribution material does not introduce processing problems during inline production of the article, and the article is simple, inexpensive and easy to manufacture, use and maintain.

Now that the preferred embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

TABLE

| | 1^{st} Time | 2^{nd} Time | 3^{rd} Time | 1^{st} Rewet | 2^{nd} Rewet | 3^{rd REWET} |
|---|---|---|---|---|---|---|
| ADL or ADS | (s) | (s) | (s) | (g) | (g) | (g) |
| 30 gsm NW | 52 | 52 | 70 | 0.1 | 9.5 | 11.4 |
| 50 gsm NW | 36 | 34 | 43 | 0.1 | 10.0 | 12.5 |
| AD | 15 | 18 | 22 | 0.1 | 5.8 | 10.8 |
| DW | 36 | 34 | 33 | 0.1 | 11.3 | 12.6 |
| AD + | 13 | 12 | 13 | 0.1 | 0.5 | 1.1 |
| 30 gsm NW | | | | | | |
| FAD + AD | 12 | 13 | 17 | 0.4 | 0.6 | 6.1 |
| DW + DW | 32 | 31 | 37 | 0.1 | 10.1 | 10.6 |
| AD + DW | 12 | 12 | 13 | 0.1 | 0.4 | 1.6 |
| DW + AD | 12 | 15 | 19 | 0.1 | 3.8 | 8.1 |

1. An absorbent article comprising:
(A) a topsheet;
(B) an absorbent core; and
(C) a layered acquisition/distribution transfer system disposed intermediate said topsheet and said absorbent core;
said system comprising at least a pair of apertured films including a first film facing said topsheet and a second film facing said absorbent core, each said film being three dimensional and defining pores which taper inwardly in a first direction from said topsheet to said absorbent core and terminate in substantially thick projections in the first direction beyond a main plane of the film, said first film having a larger average pore size than said second film.

2. The article of claim 1 wherein on average the thickness of said projections is at least 15-50 times that of the main plane of the film.

3. The article of claim 1 wherein said films have a combined thickness of at least 30 mils (0.76 mm).

4. The article of claim 1 wherein said first film has an average pore size of 0.3-10 mm in diameter, and said second film has an average pore size of 0.1-2.0 mm in diameter.

5. The article of claim 4 wherein said first film has an average pore size of 0.5-5.0 mm in diameter, and said second film has an average pore size of 0.3-1.5 mm in diameter.

6. The article of claim 5 wherein said first film has an average pore size of 1.0-2.0 mm in diameter, and said second film has an average pore size of 0.5-1.0 mm in diameter.

7. The article of claim 1 wherein said first film has a basis weight at least as high as said second film, said first film has a basis weight of 25-100 gsm, and said second film has a basis weight of 10-35 gsm.

8. The article of claim 7 wherein said first film has a basis weight of 30-65 gsm, and said second film has a basis weight of 15-30 gsm.

9. The article of claim 8 wherein said first film has a basis weight of 35-50 gsm, and said second film has a basis weight of 20-30 gsm.

10. The article of claim 1 wherein said first film has a basis weight at least as high as said second film.

11. The article of claim 1 wherein said first and second films are laminated together.

12. The article of claim 11 wherein said first and second films are contiguous.

13. The article of claim 1 wherein said first and second films are formed of substantially the same polymer.

14. The article of claim 13 wherein said same polymer is polyethylene.

15. The article of claim 1 wherein said pores are generally conical.

16. The article of claim 1 wherein said films have a combined thickness of at least 50 mils (1.3 mm).

17. The article of claim 1 wherein each of said films is formed of a wettable and substantially non-absorbent thermoplastic polymer.

18. An absorbent article comprising:
(A) a topsheet;
(B) an absorbent core; and
(C) a layered acquisition/distribution transfer system disposed intermediate said topsheet and said absorbent core;
said system comprising at least a pair of apertured films including a first film facing said topsheet and a second film facing said absorbent core, each said film being three dimensional and formed of a wettable and substantially non-absorbent thermoplastic polymer, each said film defining generally conical pores which taper inwardly in a first direction from said topsheet to said absorbent core;
said first film having a larger average pore size than said second film, said first film having an average pore size of 0.3-10 mm in diameter, and said second film having an average pore size of 0.1-2.0 mm in diameter;
said first film having a higher basis weight than said second film, said first film having a basis weight of 25-100 gsm, and said second film having a basis weight of 10-35 gsm;
said first and second films being contiguous, formed of essentially the same polymer and having a combined thickness of at least 30 mils (0.76 mm).
